(19) United States
(12) Patent Application Publication
(10) Pub. No.: US 2024/0189459 A1
(43) Pub. Date: Jun. 13, 2024

(54) METASTABLE-FREE LANTHANIDE BASED CONTRAST AGENTS AND METHOD

(71) Applicant: Inventure LLC, Southbury, CT (US)

(72) Inventors: Richard DESLAURIERS, Woodbury, CT (US); Michael MILBOCKER, Holliston, MA (US); Paul LOMBARDO, Stamford, CT (US)

(73) Assignee: Inventure LLC, Southbury, CT (US)

(21) Appl. No.: 18/537,462

(22) Filed: Dec. 12, 2023

Related U.S. Application Data
(60) Provisional application No. 63/431,950, filed on Dec. 12, 2022.

(51) Int. Cl. A61K 49/10; A61K 47/26; A61K 47/54; B01D 15/36; B01J 39/05

(52) U.S. Cl. CPC A61K 49/108; A61K 47/26; A61K 47/547; B01D 15/362; B01J 39/05

(57) ABSTRACT

A method of preparing a stable lanthanide complex substantially free of metastable lanthanide complex and free lanthanide ion includes the steps of (i) complexing a macrocyclic chelator with a lanthanide metal in a suitable solvent to obtain a first solution of the metal complex, (ii) challenging the metal complex to cause metastable complexes to form free lanthanide ion and chelate to yield a second solution, and (iii) removing the free lanthanide ion from the second solution with a separation media.

### CROSS-REFERENCE TO RELATED APPLICATIONS

The present application claims the benefit of U.S. provisional application No. 63/431,950 filed on Dec. 12, 2022, the entire disclosure of which is incorporated herein by reference.

### BACKGROUND OF THE DISCLOSURE

### 1. Field of the Disclosure

The present disclosure is directed to pharmaceutical formulations of contrast agents used in magnetic resonance imaging (MRI) and methods of making the same. The present disclosure is further directed to such pharmaceutical formulations of chelate complexes with paramagnetic metal ions as well as methods for obtaining and purifying the same. In particular, the pharmaceutical formulations are free or substantially free of metastable lanthanide complexes.

### 2. Description of Related Art

Contrast agents are used in diagnostic imaging to increase the contrast and facilitate imaging of structure and fluids in the human body.

Commonly used MRI contrast agents are based on paramagnetic lanthanide series metals, and particularly gadolinium. Complexes of chelated complexes of gadolinium include, for example, macrocyclic chelate compounds such as DOTA gadoterate (1,4,7,10-tetraazacyclo-dodecane-N, N′, N″, N′″-tetraacetic acid) and gadoteridol HPDO3A, DTPA (diethylenetriamine), and Gadopiclenol.

When first introduced into the human body, lanthanide chelate complexes are in substantial chemical equilibrium with their free ionic forms. However, over time, these chelate complexes of lanthanides release lanthanide ions into the body. Chelate complexes based on gadolinium release gadolinium ion.

The environment of the body has a destabilizing effect on these complexes, shifting the equilibrium toward the free ionic form which poses a risk of undesirable release of lanthanides, including gadolinium ion. Such free lanthanide ions in the body can be highly toxic, particularly gadolinium ion, and even in low amounts. Moreover, the release of lanthanide ions into the body is especially problematic because the administration of contrast agents is often necessary to be repeated during diagnostic tests and/or induction and monitoring of the effectiveness of therapeutic treatments, thereby compounding the risks.

There have been prior approaches for improving the tolerability of gadolinium chelate complexes. For example, one approach has been adding an excess of a chelating compound to a lanthanide complexing chelating compound to reduce the amount of free gadolinium ion in the product in the bottle prior to injection. However, the chelating compound itself is toxic. Adding excess chelate is not desirable. Further, while excess chelate can achieve a reduced gadolinium ion formation upon manufacturing and storage in a bottle prior to injection, excess chelate does not result in reduced gadolinium ion formation in the body. The excess chelate does not significantly change the equilibrium between free gadolinium ions and its chelated form. Further, the small number of gadolinium ions that are chelates with the excess chelate are metastable. When metastable chelate complexes are introduced or injected into the body, the metastable chelate complexes will readily release free gadolinium. Thus, while there can be a perceived safety enhancement with respect to in-bottle testing, such safety is not in fact realized in practice when injected in the human body. This is because reduction of gadolinium ion in the bottle does not correlate with reduction in the release of free gadolinium ion in the body. The bottle is a controlled environment. The human body is not.

In other approaches, a chelate compound such as a ligand L can be added in excess with respect to a macrocyclic chelate compound in the form of an additive having the formula X [X′, L], where X and X′ are metal ions and L is an excess of chelate compound. In this example, the metal ions are competitive metal ions, for example calcium, sodium, zinc, or magnesium. These additives are intended to capture free lanthanides by releasing the metal ions X and X to form metastable complexes with free gadolinium ions present in the formulation. While this approach reduces the toxicity of the free excess chelate, it shifts the gadolinium ion to complex equilibrium toward releasing more gadolinium ions. While the additional release of free gadolinium ions can be taken up by the excess X[X′,L], more metastable chelates result due to the destabilizing effect of competitive metal ions that are introduced. In fact, this approach highlights the fact that there are many more metastable complexes in the original formulation than the number of free gadolinium ions present.

The commonly available contrast agents are not tested for the presence and quantity of metastable complexes.

Free chelate can form more toxic complexes when introduced into the body. According to the LD₅₀ value, free macrocyclic chelate compounds (HP-DO3A, DO3A, DOTA) in the form of X [X′, L] are at least about 10 times more toxic than the free macrocycles.

The safety of gadolinium contrast agents also depends on the purity of the gadolinium used. Gadolinium found in nature is associated with other heavy metals that are toxic and provide little to no benefit for diagnostic imaging.

MRI contrast agents that are based on gadolinium commonly also traces of Scandium (Sc), Yttrium (Y), Lanthanum (La), Cerium (Ce), Praseodymium (Pr), Neodymium (Nd), Europium (Eu), Terbium (Tb), Thulium (Tm), Dysprosium (Dy), Holmium (Ho), and Erbium (Er). In an analysis, Tb, Tm, Eu, and La were, on average, found in the highest amounts, 0.42 mg/L, 0.17 mg/L, 0.17 mg/L, and 0.16 mg/L, respectively.

Accordingly, it has been determined by the present disclosure that there is a continuing need for compositions and methods for preparing compositions of lanthanide metal complexes with macrocyclic chelators which do not contain, or only contain small amounts of free lanthanide metal ion and do not contain, or only contain small amounts of metastable lanthanide metal complexes which release free lanthanide metal ion when introduced into a mammalian body and that overcome, alleviate, and/or mitigate one or more of the aforementioned and other deleterious effects of prior attempts.

### SUMMARY OF THE DISCLOSURE

The present disclosure provides compositions and methods that are particularly suitable for pharmaceutical production on an industrial scale, including such compositions and methods where stoichiometric balancing of metal ion to chelator is obtained in the final product using a separation media, including ionic acceptor resins and selectively permeable membranes. Other separation media are contemplated, including, mixed-mode cation exchange, mixed-mode anion exchange cation exchange chromatography, anion exchange chromatography and monoliths chromatography.

The present disclosure further provides methods and compositions of the lanthanide metal complexes that are particularly well suited for providing MRI contrast agents containing such formulations.

The present disclosure still further provides methods for purifying lanthanide compounds, methods for removing free lanthanide ions from a complex formed from the purified lanthanide compound, and stable lanthanide complexes substantially free of metastable lanthanide complexes and free lanthanide ions. For example, a lanthanide complex formed by the chelation of gadolinium ions with DOTA or Diethylenetriamine-N-oxide pentaacetic acid-bisamide.

The term “substantially free of metastable lanthanide complexes” means that metastable lanthanide complexes are less than 0.5%, preferably less than 0.4%, more preferably less than 0.1%, and still more preferably less than 0.05% by volume of non-metastable lanthanide metal complexes in the lanthanide metal complex. The percentage of metastable complexes can be determined by Ultraviolet (UV) spectroscopy.

The term “substantially free of lanthanide ions” means that free lanthanide ions comprise less than 5 ppm, preferably less than 3 ppm, more preferably less than 1 ppm, and still more preferably less than 0.1 ppm by weight of lanthanide metal complexes. Lanthanide ions can include gadolinium.

A solid phase ionic acceptor as discussed herein can be a resin-bound ionic acceptor or a resin-bound acceptor chelator, and at least one of the ionic acceptors can be in the form of a meglumine salt. The present disclosure has found that this has the significant advantage that meglumine ions instead of sodium ions are released after the formation of the lanthanide complex. This also has the advantage that the content of sodium ions in the resulting lanthanide metal complex is minimized.

The solid phase ionic acceptors can be recycled after being used to regenerate a material suitable for the manufacture of pharmaceutical formulations of lanthanide complexes.

The present disclosure still further provides a process for preparing macrocyclically chelated lanthanide metal complex formulations that contain less than 0.5%, preferably less than 0.4%, more preferably less than 0.1%, still more preferably less than 0.05%, and most preferably 0% by volume of non-metastable lanthanide metal complexes in chelated lanthanide metal complex, and less than 5 ppm, preferably less than 3 ppm, more preferably less than 1 ppm, and still more preferably less than 0.1 ppm by weight of lanthanide metal complexes. In certain embodiments, the formulations have a slight excess of free chelator.

A method according to the present disclosure uses solid phase acceptor chelators to remove excess lanthanide metal ions after the macrocyclically chelated lanthanide metal complex is challenged.

Examples of challenging include exposure to a solid bound chelator, strong acid cation resin, or weak cation resin but could also include heating, altering pH, isotope degradation by allowing a prolonged period of time such as 24 hours to pass, exposure to alternating magnetic field, introducing a competing chelating agent or ligand, ultrasonication, and exposure to electromagnetic radiation.

For example, heating can provide the energy required to overcome the activation barrier, allowing the metastable complex to transition to a more stable state or dissociate. By altering the pH, the metastable complex can be caused to transition to a more stable form or caused to dissociate. Competing chelating agent or ligand can displace the original chelating agent or ligand from the metastable complex, causing destabilization.

In embodiments, the method uses a solid phase acceptor resin to remove excess chelate prior to adding a certain amount of excess of chelator.

The present disclosure has found that the methods discussed herein are particularly suited to industrial synthesis of macroscopically chelated lanthanide metal complex formulations because the methods are not dependent upon stoichiometric introduction of raw materials.

The present disclosure also provides a method for the preparation of MRI contrast agents suitable for these methods.

The present disclosure has found that treatment of the lanthanide complex with an anionic or cationic acceptor resin is effective to remove coordinated and uncoordinated metal ion impurities from the lanthanide complex solution by, for example transmetalation. These impurities can include calcium, manganese, zinc, copper, and magnesium.

The solid phase ionic acceptor can be a resin-bound ionic acceptor or a resin-bound acceptor chelator, and at least one of the ionic acceptors can be in the form of a meglumine counter ion. Advantageously, meglumine ions instead of sodium ions are released during ionic exchange. Thus, the content of sodium ions in the resulting lanthanide metal complex can be minimized.

The present disclosure has further found that solid phase ionic acceptors can be recycled after use to regenerate a material suitable for the manufacture of pharmaceutical formulations of lanthanide complexes.

As used herein, the term “ionic acceptor” refers to any solid agent that forms a complex or compound with an uncomplexed moiety of a lanthanide complex formulation. Uncomplexed moieties include chelators such as DOTA and free lanthanide metal ions such as Gd³⁺, and other 2+ and 3+ metal ions.

The term “chelating agent” refers to ligands that form metal coordination complexes that contain a plurality of metal donor atoms arranged so that 5, 6, or 7-membered coordination rings are obtained. Coordination rings preferably have an uncoordinated backbone of carbon atoms or uncoordinated heteroatoms linking donor metal atoms. Chelating agents are chemical compounds that react with metal ions to form a more stable, water-soluble complex. Chelating agents are also known as chelants, chelators, or sequestering agents. Chelating agents have a ring-like center which forms at least two bonds with the metal ion allowing it to be excreted. As used herein, a chelating agent has at least two functional groups which donate a pair of electrons to the metal, such as ═O, —NH2 or —COO⁻. Furthermore, these groups must be located to allow ring formation with the metal. Chelating agents are widely found in living systems and are of importance in cellular metabolism.

The term “macrocyclic” has its ordinary and customary meaning in the field of coordination chemistry and refers to a chelator in which at least some of the chelator's metal donor atoms are covalently bonded as part of a ring system.

The expression “chelator in uncomplexed form” refers to “free chelator”, that is, without any coordinated metal ions. A chelator in uncomplexed form does not contain any coordinated lanthanide or other metal ions and is thus fully available for subsequent metal complex formation. The “chelator in uncomplexed form” can contain metal ions in ionic form, for example, when it is present as salts of a metal donor group, for example, a carboxylic acid.

The expression “metastable lanthanide complex” refers to a lanthanide complex wherein the metal ion is not fully contained within the coordination ring. A “metastable lanthanide complex” has a less stable kinetic stability constant compared to stable or fully coordinated lanthanide complexes that are referred to herein as non-metastable.

The term “solid phase-bound acceptor” refers to an ionic or chelating agent covalently bound to a solid phase material that is insoluble in the solvent used to form the lanthanide complex. The bound acceptor forms complexes with free metal ions in solution and is thus capable of removing or “accepting” any such metal ions from solution. Preferably, the acceptor is chosen to be different from the “macrocyclic chelator” and therefore advantageously has a lower formation constant for the lanthanide metal than the “macrocyclic chelator” and is accordingly chosen so that it cannot displace the lanthanide metal ion from the lanthanide complex.

In some embodiments according to the disclosure, either alone or together with any one or more of the aforementioned and/or after-mentioned embodiments, a method of preparing a stable lanthanide complex substantially free of metastable lanthanide complex and free lanthanide ion comprises (i) complexing a macrocyclic chelator with a lanthanide metal in a suitable solvent to obtain a first solution of the metal complex; (ii) challenging the metal complex to cause metastable complexes to form free lanthanide ion and free chelate to yield a second solution; and (iii) removing the free lanthanide ion and free chelate from the second solution with a separation media.

In some embodiments according to the disclosure, either alone or together with any one or more of the aforementioned and/or after-mentioned embodiments the separation media is an ionic acceptor resin that is cationic, and the method further comprises: (iv) separating the ionic acceptor resin from the second solution to obtain a third solution that contains a metal complex containing less than 5 ppm free lanthanide ion and less than 0.5% metastable lanthanide complexes.

In some embodiments according to the disclosure, either alone or together with any one or more of the aforementioned and/or after-mentioned embodiments, the method further comprises: (v) removing excess chelate from the third solution by contacting one or more times with an anionic acceptor resin, whereby the excess chelate forms a complex with the anionic acceptor resin.

In some embodiments according to the disclosure, either alone or together with any one or more of the aforementioned and/or after-mentioned embodiments, the method further comprises: (vi) adding a chelator in a non-complexed form in the range from 0.005 to 0.05% w/v to the second or third solution to obtain a liquid pharmaceutical composition.

In some embodiments according to the disclosure, either alone or together with any one or more of the aforementioned and/or after-mentioned embodiments, the (ii) challenging further comprises adding an amount of meglumine to the first solution or cooling to room temperature at a pH below 2.5 and for a time of 2 hours to 24 hours.

In some embodiments according to the disclosure, either alone or together with any one or more of the aforementioned and/or after-mentioned embodiments, the (iii) removing is performed at a pH from 4.0 to 6.0.

In some embodiments according to the disclosure, either alone or together with any one or more of the aforementioned and/or after-mentioned embodiments, the (iii) removing is by contacting with an ionic acceptor resin to cause excess lanthanide to form a complex with the ionic acceptor resin.

In some embodiments according to the disclosure, either alone or together with any one or more of the aforementioned and/or after-mentioned embodiments, the ionic acceptor resin is selected from the group consisting of: iminodiacetic acid, ethylenediaminetetraacetic acid and diethylenetriaminepentaacetic acid, thiourea, strong acid cation, and weak acid cation.

In some embodiments according to the disclosure, either alone or together with any one or more of the aforementioned and/or after-mentioned embodiments, the (iii) removing excess lanthanide is by a membrane separation that prevents passage of chelator and lanthanide complex through the membrane, a protein-based affinity chromatography process, or reverse osmosis.

In some embodiments according to the disclosure, either alone or together with any one or more of the aforementioned and/or after-mentioned embodiments, the lanthanide metal is at least one metal selected from the group consisting of: gadolinium, praseodymium, dysprosium, europium, and thulium and/or the macrocyclic chelator is at least one macrocyclic chelator selected from the group consisting of: DOTA, NOTA, DO3A, BT-DO3A, HP-DO3A and PCTA.

In some embodiments according to the disclosure, either alone or together with any one or more of the aforementioned and/or after-mentioned embodiments, the method further comprises: alternating a magnetic field at a temperature below the Curie Temperature of the lanthanide metal during the (i) complexing.

A method for removing metastables from a macrocyclic chelator and lanthanide metal complex, the method comprises: providing a solution containing the macrocyclic chelator and lanthanide metal complex; challenging the metal complex to cause metastable complexes to form free lanthanide ion and chelate to yield a solution; removing excess lanthanide metals and macrocyclic chelator from the solution with a separation media that comprises a cationic or anionic acceptor resin configured with a highest affinity toward 3+ ions; and separating the cationic or anionic acceptor resin from the solution to obtain a second solution that contains a metal complex substantially free of lanthanide ion and metastable lanthanide complexes.

In some embodiments according to the disclosure, either alone or together with any one or more of the aforementioned and/or after-mentioned embodiments, the method further comprises: adjusting a pH of the solution to be suitable for injection in a human body with meglumine addition after the separating.

In some embodiments according to the disclosure, either alone or together with any one or more of the aforementioned and/or after-mentioned embodiments, the method further comprises: the challenging comprises: applying the solution to an ion exchange resin configured to remove free lanthanide metals and dechelate metastable complexes from the solution for at least 5 minutes and at most 30 minutes.

In some embodiments according to the disclosure, either alone or together with any one or more of the aforementioned and/or after-mentioned embodiments, the ion exchange resin is a cation exchange resin.

In some embodiments according to the disclosure, either alone or together with any one or more of the aforementioned and/or after-mentioned embodiments, the ion exchange resin is a chelating resin selected from the group consisting of: thiourea, iminodiacetic acid, animinophosphonic acid, ethylenediaminetetraacetic acid and diethylenetriaminepentaacetic acid.

In some embodiments according to the disclosure, either alone or together with any one or more of the aforementioned and/or after-mentioned embodiments, the lanthanide metal chelate complex is gadoteric acid.

In some embodiments according to the disclosure, either alone or together with any one or more of the aforementioned and/or after-mentioned embodiments, a method for removing metastables from a macrocyclic chelator and lanthanide metal complex comprises: controlling a temperature to be in a range of 10° C. to 50° C. while applying the solution to the ion exchange resin.

In some embodiments according to the disclosure, either alone or together with any one or more of the aforementioned and/or after-mentioned embodiments, a method for removing metastables from a macrocyclic chelator and lanthanide metal complex comprises: applying the solution to the ion exchange resin in concentration of 0.1 to 1.0 equivalents.

In some embodiments according to the disclosure, either alone or together with any one or more of the aforementioned and/or after-mentioned embodiments, an injectable contrast agent solution for use in diagnostic imaging comprises a complex of a macrocyclic chelate and a lanthanide metal; less than 0.5% metastable lanthanide complexes by volume of non-metastable lanthanide metal complexes in the lanthanide metal complex; and less than 5 ppm free lanthanide ions by weight of lanthanide metal complexes.

The present disclosure also provides a product. The product includes a container, a sequestering or destabilizing agent, and a solution of Gd-DOTA complex.

The solution of Gd-DOTA complex has a percentage of fully chelated DOTA and gadolinium complexes that is at least 100%, at least 95%, or at least 90%. This solution can be ready for a final destabilizing and sequestering. The sequestering or destabilizing agent can be one of the separation media discussed herein.

The product would encapsulate the unwanted byproducts in a stable matrix or resin to prevent the release of unwanted metastable intermediates.

The container can be a vial, a drum, or other known container suitable for storing and transporting contrast agents.

The present disclosure also provides a method that includes destabilizing fully chelated DOTA and gadolinium complexes, either alone or together with any one or more of the aforementioned and/or after-mentioned embodiments.

Although the present disclosure is discussed with respect to lanthanide metals like gadolinium, it is contemplated that the present disclosure is also applicable to complexes having manganese in place of the lanthanide due to its paramagnetic properties.

The above summary is not intended to describe each disclosed implementation, as features in this disclosure can be incorporated into additional features as detailed herein below unless clearly stated to the contrary.

### DESCRIPTION OF THE DRAWINGS

The accompanying drawings illustrate aspects of the present disclosure, and together with the general description given above and the detailed description given below, explain the principles of the present disclosure. As shown throughout the drawings, reference numerals designate like or corresponding parts.

FIG. 1 is a process flow chart of a method according to the present disclosure.

FIG. 2 shows a structure of DOTA.

FIG. 3 shows a structure of Diethylenetriamine-N-oxide pentaacetic acid-bisamide.

FIG. 4 shows a membrane purification system according to the present disclosure.

FIG. 5 is a process flow chart of a method according to the present disclosure.

FIG. 6 is a graph showing test data according to the present disclosure.

FIG. 7 is a graph showing additional test data according to the present disclosure.

### DETAILED DESCRIPTION OF THE DISCLOSURE

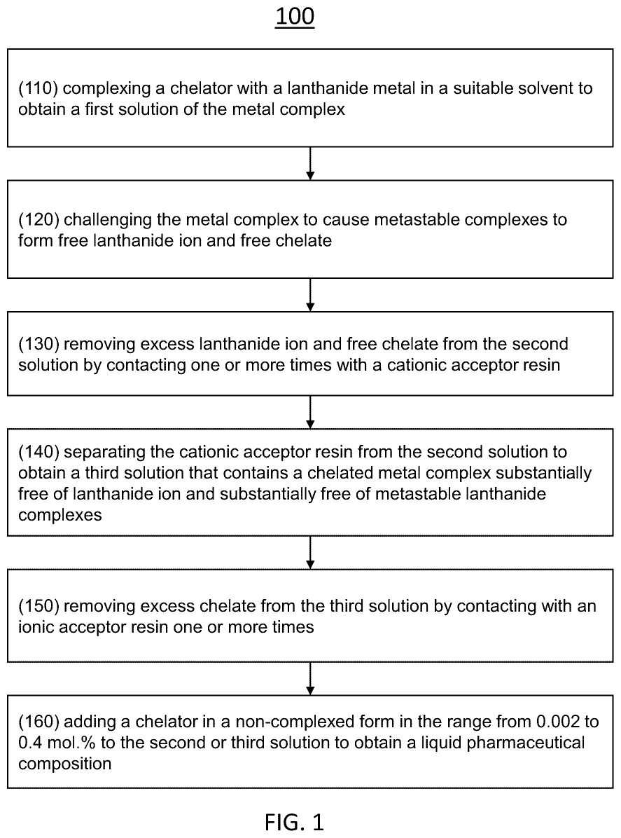

Referring to FIG. 1, there is shown a process 100 according to the present disclosure for preparing a stable lanthanide complex substantially free of metastable lanthanide complex and free lanthanide ion. The process includes the steps of: (110) complexing a chelator with a lanthanide metal in a suitable solvent to obtain a first solution of the metal complex, (120) challenging the metal complex to cause metastable complexes to form free lanthanide ion and free chelate in a second solution while (130) removing excess lanthanide ion and free chelate from the second solution by contacting one or more times with a cationic or anionic acceptor resin, whereby the excess lanthanide and the free chelate form complexes with the cationic acceptor or anionic resin, and (140) separating the cationic or anionic acceptor resin from the second solution to obtain a third solution that contains a chelated metal complex substantially free of free lanthanide ion and substantially free of metastable lanthanide complexes.

Between step (110) and (120), or in step (120) or in step (140), optionally, there is an addition of meglumine. As an alternative to meglumine, N-sugar alcohols and meglumine equivalents are contemplated.

The process can further include (150) removing excess chelate from the third solution by contacting with an anionic acceptor resin one or more times, whereby the excess chelate forms a complex with the anionic acceptor resin. If the chelate is protonated a cation resin can be used.

The process can further include (160) adding a chelator in a non-complexed form in the range from 0.002 to 0.4 mol. % to the second or third solution to obtain a liquid pharmaceutical composition.

In an example embodiment, the lanthanide metal is gadolinium. In other example embodiments, the lanthanide metal is praseodymium, dysprosium, europium, or thulium.

In an example embodiment, the macrocyclic chelator contains one or more of DOTA, NOTA, DO3A, BT-DO3A, HP-DO3A and PCTA.

In an example embodiment, in combination with the foregoing, the gadolinium complex formed does not contain any metastable gadolinium complexes and the solution containing the gadolinium complex does not contain any free gadolinium ion.

In another example embodiment, in combination with the foregoing, the gadolinium complex formed less than 0.5%, preferably less than 0.4%, and more preferably less than 0.1%, and still more preferably less than 0.05% metastable gadolinium complexes by volume of non-metastable gadolinium complexes and the solution containing the gadolinium complex less than 5 ppm, preferably less than 3 ppm, more preferably less than 1 ppm, and still more preferably less than 0.1 ppm free gadolinium ion by weight (mg/kg of final API solution).

In further example embodiments, in combination with the foregoing, the solution containing the gadolinium complex does not contain any chelator in its uncomplexed form.

In further example embodiments, in combination with the foregoing, the solution containing the gadolinium complex contains an excess of 0.005-0.050% w/v chelate which does not contain coordinated metal ions.

The acceptor resins are solid phase bound. In an example, the cationic acceptor chelator is has a strong acid functional group with a meglumine counter ion. Additionally, the lanthanide complex solutions are formulated to produce an MRI contrast agent. An anionic acceptor chelator is also contemplated.

In an example embodiment the solid phase chelator is iminodiacetic acid. In another example, the solidphase chelator is a thiourea chelator resin.

The method and pharmaceutical compositions of the disclosure relate to the use of the solid phase chelators to remove metastable lanthanide complexes and optionally uncomplexed chelate to obtain a pharmaceutical composition with a precise amount of uncomplexed chelate that is not sensitive to the stoichiometric amount of raw ingredients used in an industrial process for making the pharmaceutical composition.

Acceptors according to the present disclosure can be acceptor chelators or ionic exchange acceptors chosen so that the kinetics of capture of the free metal ion in solution is fast. For this reason, linear (i.e., non-macrocyclic) acceptor chelators are preferred. Acceptor chelators are to be assR30ociated with a solid phase so that the acceptor chelator is easily separated from the lanthanide complex solution with which it is in contact with by filtration. Suitable solid phase materials include synthetic polymers and copolymers.

Acceptors according to the present disclosure are preferably bound to a solid state. Alternatively, the lanthanide complex solution and the acceptor solution can be separated by a semipermeable membrane which allows free chelator and/or free metal ion through to the acceptor but does not allow both the lanthanide complex and the acceptor to pass through.

Suitable solvents for complex formation in step (110) are known in the art. Water is the preferred solvent. The formation of the lanthanide complex using macrocyclic chelators is a multi-step process that involves a metastable initial complex that very slowly coordinates to a final and more thermodynamically stable metal complex. In step (110), it is preferred to ensure that the lanthanide complex is formed before proceeding to step (120).

Interacting the lanthanide complex formed in step (110) with an acceptor in step (130) is suitably carried out so that the entire “first solution” is exposed to the acceptor. For example, the acceptor can be a chelator resin or cation exchange resin.

Removal of free lanthanide ions can be achieved by several processes. In the first process, the solid phase resin is mixed with the solution of lanthanide complex of step (110). In a second process, the solid phase can be provided as a column and the lanthanide complex solution eluted through the column. In a third option, the solution of lanthanide complex of step (110) can be separated from a solution of high molecular weight chelator by a membrane that prevents passage of chelator and lanthanide complex. The third option is advantageous if the acceptor is not solid bound.

Removal of free lanthanide ions can also be achieved with combinations processes discussed in the preceding paragraph.

Separation of the acceptor from the lanthanide complex solution in step (140) can be carried out either by filtering the solution to remove the lanthanide-bound resin or by collecting the eluate from the elution column, respectively.

The addition in step (140) can be performed without prior in-process analysis of the concentration/amount of free lanthanide ion in either the first or second solution.

If the initial charge of raw materials is stoichiometric, then removal of free gadolinium ions will result in free chelator. The amount of free chelator can be assessed by several methods known in the art, including for example, Performance Liquid Chromatography (HPLC), mass spectrometry, and titration.

Alternatively, additional lanthanide can be added until a slight excess of free lanthanide ion is detected and quantified via xylenol orange assay. The final slight excess of free lanthanide is then eliminated by a stoichiometric amount of chelator. In this context, slight means greater than 10⁻⁶, preferably greater than 10⁻⁹.

The free chelator can be added either as a solid or as a solution. In preferred embodiments, free chelator is added as a solution to facilitate the procedure and increase accuracy.

When a macrocyclic chelator solution is prepared for step (110), an advantageous method is to charge the reaction vessel with a suitable volume fraction from a stock solution prior to addition of the lanthanide. The chelator stock solution can conveniently be used for addition in optional step (150).

In some instances, the chelator has a low purity, and the chelator impurity is a chelate complex with a metal other than the desired lanthanide series metal, for example calcium. In such instances, the chelator can be purified prior to use in the formation of the lanthanide complex by exposing the chelate to a solid state acceptor. In this context, low purity means less than 99.0% of the chelate, preferably less than 99.5% of the chelate, and most preferably 99.9% of the chelate.

In other example embodiments, an excess lanthanide can be introduced in step (110) to promote displacement of any coordinated metal impurity from the chelator, where the displaced metal impurity has a lower complexation constant with the macrocyclic chelator than the lanthanide. The formation of the lanthanide complex in step (110) is performed by heating the reaction mixture, so that the thermodynamic product, the lanthanide metal complex, is favored over the coordinated metal impurity. Subsequently, the metal impurity and free lanthanide is removed in step (140). This has the advantage that the need for a separate purification step of the macrocyclic chelator can be eliminated.

The present disclosure has found that the existence of metal impurity complexes in the lanthanide complex solution can be responsible for an enigmatic result often seen regarding commercial gadolinium-DOTA complexes. Without wishing to be bound by a particular theory, it is believed that the presence of excess chelate should ensure complexation of free gadolinium ions which occurs even in formulations synthesized in stoichiometric ratio due to the kinetic stability constant. However, since the coordination of gadolinium involves three oxygen atoms residing on DOTA and the coordination of calcium involves two oxygen atoms, it is considered that the calcium-DOTA complex is less stable than the gadolinium-DOTA complex. This can result in an equilibrium concentration of free gadolinium that is not reduced by addition of excess chelate.

The persistence of free gadolinium ion even in the presence of free chelate can occur because the free gadolinium assay is based on xylenol orange chelate and involves the lanthanide solution remaining in contact with the chelator xylenol orange for a significant period of time prior to measurement by UV absorption. Thus, the calcium ion, being relatively mobile, continues to participate in transmetalation in the presence of the competitive xylenol orange chelator, now representing a large chelator excess relative to any intention DOTA excess. The xylenol orange assay consequently measures free gadolinium ions as a function of the calcium impurity.

The present disclosure has found that an intentional slight excess of gadolinium ensures complete formation of DOTA complexes to form Gd-DOTA, and DOTA-coordinated impurity metal ions (such as calcium) can be efficiently trans metallized into solution as free ions due to less favorable thermodynamic stability. Consequently, excess gadolinium can be used as a destabilizer of the Ca-DOTA complex in step (120) so that both free calcium and gadolinium ions can be eliminated in step (130).

Table 1 below lists various DOTA complexes with metal ions and their thermodynamic stability.

TABLE 1

| Ion | Stability Constant |
|---|---|
| Fe⁺³ | 29.4 |
| Dy⁺³ | 24.8 |
| Gd⁺³ | 24.7 |
| TM⁺³ | 24.4 |
| Eu⁺³ | 23.5 |
| Pr⁺³ | 23 |
| La⁺³ | 22.9 |
| Cu⁺² | 22.3 |
| Zn⁺² | 20.8 |
| Mn⁺² | 20 |
| Ca⁺² | 17.2 |
| Mg⁺² | 11.9 |
| Na⁺¹ | 4.2 |
| K⁺¹ | 1.6 |

In Table 1, the various thermodynamic stability constants are given for metal-DOTA complexes. In coordination chemistry, a stability constant is an equilibrium constant for the formation of a complex in solution. It is a measure of the strength of the interaction between the reagents that come together to form the complex. The data of Table 1 is from: Popov, K. Felcman, J. Delgado, R. Arnaud-Neu, F. Anderegg, G. Pure Appl. Chem. 77, 8, 2005; Cacheris, W P, Nickel, S K Sherry, A D; inorg. Chem. 2646, 1986; Toth, E. Brucher, E, Inorganica Chimica Acta, 221(1-2), 1994, pp. 165-167.

Therefore, coordinated metal ions typically found in DOTA and gadolinium oxide have a lower stability constant than Gd-DOTA (such as calcium, manganese, zinc, copper, or magnesium), are released by Gd transmetalation, and are then removed with an acceptor chelator in steps (130) and (140).

The excess of lanthanide metal in step (110) is preferably 100 to 10,000 mol. ppm, more preferably 100 to 1000 mol. ppm, and most preferably 500 to 5000 mol. ppm.

When a chelator resin is used, the chelator resin preferably has a higher thermodynamic stability constant for gadolinium than for the impurity metals. Therefore, an excess of gadolinium in the complex formation reaction in step (110) will result in a less efficient impurity removal process, since a large molar excess of acceptor resin would have to be added. The excess receptor resin preferentially removes the free gadolinium and then removes free metal impurity ions. Therefore, it is advantageous to use a small molar excess of gadolinium in the complex formation reaction in step (110).

If the macrocyclic chelator is believed to contain coordinated calcium or similar metal ions as described above, then a very low excess of gadolinium in the range of about 100 to 1000 mol ppm is preferred. This range is low enough to provide sufficient acceptor activity to remove metal impurity, but high enough to allow complete transmetalation of any impurity metal-DOTA complex.

The lowest levels of excess lanthanide metal are to be achieved by incrementally adding aliquots of such lanthanide until a positive result is observed via xylenol orange assay when tested for the presence of free lanthanide ions. If the free gadolinium assay indicates a lack of free gadolinium, additional gadolinium is to be added.

In the method of lanthanide metal complex formation, the chelator in non-complexed form is preferably in an amount in the range from 200 to 2000 mol. ppm, more preferably from 500 to 2000 mol. ppm, and most preferably from 1000 to 1500 mol. ppm relative to the macrocyclic lanthanide complex.

The chelator in its uncomplexed form can be made suitably free of impurity metal ions using an acceptor resin prior to introduction into step (110) of the method. In this context, suitably free means 10⁻⁶, preferably 10⁻⁷, more preferably 10⁻⁹.

The lanthanide metal can preferably be gadolinium, praseodymium, dysprosium, europium, or thulium.

The macrocyclic chelator can be a heptadentate or octadentate and more preferably contains N and/or O donor atoms. The donor atoms are preferably provided by carboxylate, amino, alcohol or phosphonate donor groups. The macrocyclic chelator is preferably an aminocarboxylic acid type. When the macrocyclic chelator is of the aminocarboxylic class, such chelators preferably contain DOTA, NOTA, DO3A, BT-DO3A, HP-DO3A, PCTA or Diethylenetriamine-N-oxide pentaacetic acid-bisamide.

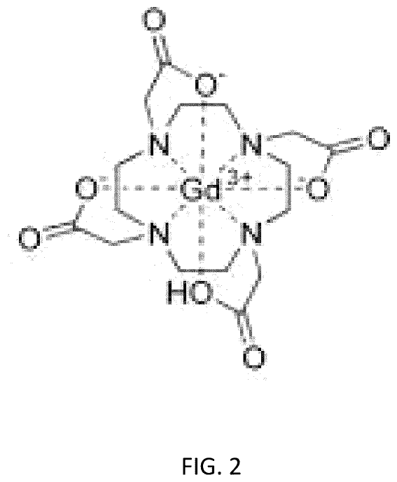

The macrocyclic chelator is preferably DOTA or a salt thereof, which is shown in FIG. 2. DOTA and its metal complexes in biomedical imaging have been described by Stasiuk and Long [Chem. Comm., 49, 2732-2746 (2013)].

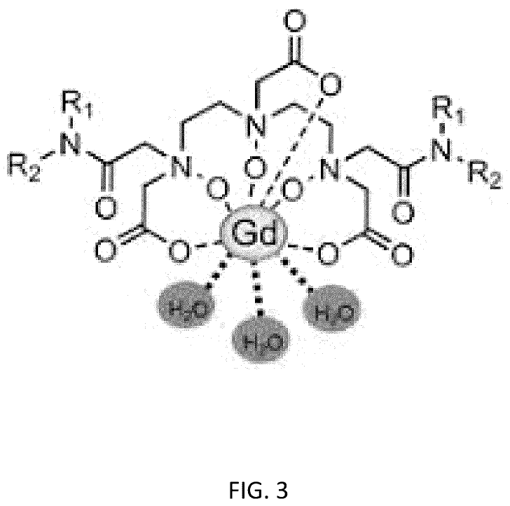

In example embodiments, the macrocyclic chelator contains Diethylenetriamine-N-oxide pentaacetic acid-bisamide, shown in FIG. 3.

In certain preferred embodiments, the lanthanide metal is gadolinium, and the macrocyclic chelator contains DOTA. Preferably, the gadolinium-DOTA complex contains the gadolinium-DOTA meglumine salt.

The acceptor chelator resin can contain thiourea, iminodiacetic acid (IDA), ethylenediaminetetraacetic acid (EDTA) or diethylenetriaminepentaacetic acid (DTPA). IDA or thiourea is preferred. Preferred solid phase acceptor chelator resins commercially available include 1) Chelex®-100 which is a styrene-divinylbenzene copolymer that is iminodiacetic acid functionalized and 2) Puromet™ MTS9140 which is a polystyrenic macroporous thiourea resin. However, strong acid cation resins such as Ag® 50W-X8/12, H+ and Purolite® C150, H+ are also preferable. Ag® 50W and Purolite® C150 are polystyrene strong acid resin crosslinked with divinylbenzene. The functional group is sulfonic acid. Purolite® C150 is macroporous and Ag® 50W is microporous. An ultrapure equivalent of Purolite® C150 is UltraClean™ UCW9126, H+. Chelex®-100 is commercially available as either the sodium or ammonium salt from Bio-Rad Laboratories Inc. Ag® 50W is commercially available as hydrogen-form resin from Bio-Rad Laboratories Inc. Purolite® C150, UltraClean™ UCW9126, and Puromet™ MTS9140 are commercially available from Purolite Company in the H+ and Na+ forms.

The cation exchange resins are selected to have a higher affinity for for certain cations over others, namely 3+ ions over 2+ ions over 1+ ions.

A neutral resin can exhibit selective ion exchange properties in the same way as an anion or cation exchange resin.

Appropriate amounts of resin needed to remove a given amount of metal are known in the art. In the case of cation exchange resin, they have no affinity for lanthanide metal complexes that are negatively charged, such as Gd(DOTA). Advantageously, there is minimal non-specific anchoring of such complexes to the solid phase.

The process for the formation of the lanthanide complexes of step (110) is typically multi-step in nature, involving multiple additions of gadolinium until a slight excess is obtained. The gadolinium must be in ionic form when reacted with chelate. Typically, gadolinium oxide is used, which requires a low pH, about 2, to ionize the Gd₂O₃ to obtain free gadolinium ions in solution.

At this low pH of about 2, the carboxylate groups of aminocarboxylate chelators (e.g., DOTA) are unable to fully complex with all gadolinium ions because the carboxylate groups are partially protonated. This leads to the formation of metastable complexes. These metastable complexes possess a much lower thermodynamic stability constant, and consequently readily release gadolinium ions into the body when challenged by competing metal ions, such as calcium, which is abundant in the body.

The pH is then raised by the addition of a base to allow for injection into a body. Preferably, the base is meglumine which can also promote the formation of the carboxylate anion, which in turn promotes the formation of the metal complex. It has been found that this has the advantageous effect of stabilizing in the bottle the complexes formed, including the metastable complexes.

Formation of the initial Gd-DOTA complex cannot be performed in the presence of meglumine. Thus, a long reaction time in step (110) is required even after a free gadolinium assay indicates the target free gadolinium ion concentration is achieved. The present disclosure has found this is because the assay does not detect gadolinium ions residing on metastable complexes. Within about 2 hours, almost all the complexes formed in step (110) are metastable. These metastable complexes mature very slowly to give the final Gd-DOTA complex with high thermodynamic and kinetic stability.

In the methods of the present disclosure, the use of meglumine in step (110) is to be avoided because the pH must remain low in order to solubilize all the gadolinium oxide and allow the complex intermediate to properly form.

After formation of the lanthanide chelate complex in step (110) the complex can be challenged in step (120) by cooling to room temperature at low pH, preferably at about pH 2, and allowing to set for 2 to 12 hours.

In step (130), the removing is preferably carried out at pH 4.0-60, more preferably 4.5-5.5, and most preferably a pH of about 5 being ideal. Here, about means+/−0.1.

In step (150), the adding is preferably performed first by adding excess chelator, then neutralizing to neutral pH of about 7.0-7.4. Here, about means+/−0.2, preferably, +/−0.1, and most preferable +/−0.05. When the desired lanthanide macrocyclic complex is a meglumine salt, this neutralization is preferably and advantageously performed using meglumine.

In preferred embodiments, a solid phase acceptor resin is present as a meglumine salt of the acceptor resin. Preferably the acceptor resin contains IDA.

Such meglumine resins have the advantage that when meglumine salts of the lanthanide metal complex are prepared, the sodium/salt ion content of the product is reduced. Thus, the chelate acceptor counterion in Chelex®-100 industrial resin is sodium, and therefore for every gadolinium ion captured, three sodium ions are released into the reaction mixture.

After use, the acceptor chelate resin containing bound metal ions can be regenerated, if necessary, for later reuse by treatment with an excess of meglumine or other counterion.

Removal of the gadolinium from the acceptor chelator can provide a useful measure of the number of metastable complexes removed from the first solution of step (110). This information can be used to add additional gadolinium to react with the excess chelator formed by removal of metastable gadolinium if use of an anionic acceptor is not used in step (150).

### EXAMPLES

### Example 1: Removing Metastables Complexes from a Macrocyclic Chelate and Lanthanide Metal Complex

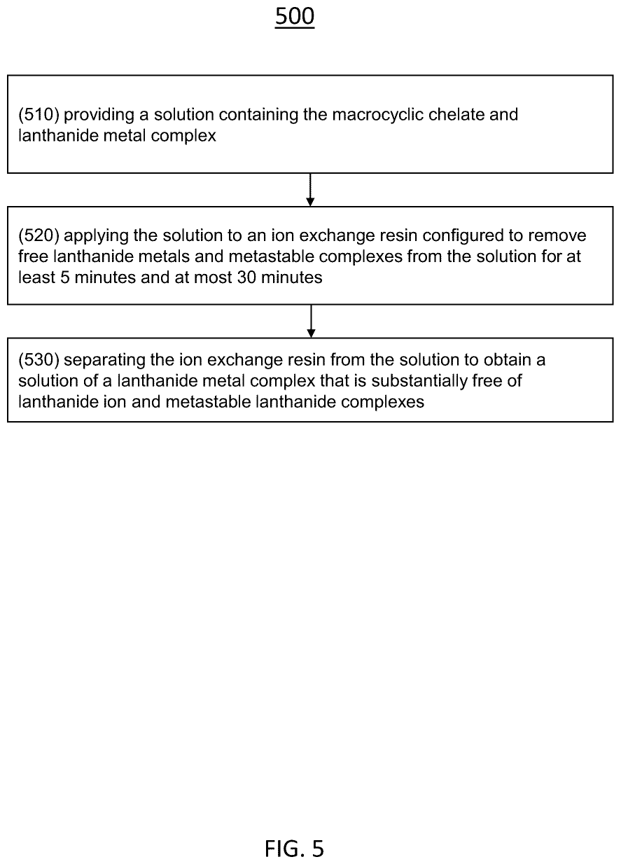

FIG. 5 shows a process 500 for removing metastables complexes from a macrocyclic chelate and lanthanide metal complex.
In step 510, a solution containing the macrocyclic chelate and lanthanide metal complex is provided.
In step 520, the solution is applied to an ion exchange resin configured to remove free lanthanide metals and metastable complexes from the solution for at least 5 minutes and at most 30 minutes. Suitable ion exchange resins are discussed herein.
In step 530, the ion exchange resin is separated from the solution to obtain a solution of a lanthanide metal complex that is substantially free of lanthanide ion and substantially free of metastable lanthanide complexes.

Resins listed in Table 2 below were selected for testing in accordance with the methods of the present disclosure.

TABLE 2

| | Counterion | | |
|---|---|---|---|
| Resin | Added | Porous | Type |
| Purolite ®C150 | meglumine | micro | Strong acid cation |
| Ag ® 50W-X8 | H+ | micro | Strong acid cation |
| Puromet ™MTS9140 | n/a | macro | Thiourea chelator |
| Purolite ®C150 | Na+ | macro | Strong acid cation |
| Ag ® 50W-X12 | H+ | micro | Strong acid cation |
| Ag ® 50W-X8 | meglumine | micro | Strong acid cation |
| Chelex ®-100 | n/a | macro | Iminodiacetic acid |

The processes of these tests were conducted at room temperature. It is contemplated that higher temperatures will also be effective, for example 25° C., 30° C., 40° C., or even 50°. Lower temperatures are also possible. It is believed that higher temperatures will increase the ion exchange rate.

For these tests, resin concentration 0.1-1.0 equivalents of resin per equivalent of gadoterate or gadolinium.

The resins were not conditioned for these tests. However, it is contemplated that conditioning can be performed.

Conditioning the resin can be performed as follows. First, the resin is washed or pre-rinsed with a suitable solvent or solvent mixture to remove any residual manufacturing impurities, dust, or other contaminants. Next, the resin is pre-swelled or hydrated by soaking the resin in an appropriate solvent or water. Then, the resin is treated with acid or base solutions to remove metal ions, metal oxides, or other inorganic impurities. A complexing agent or a regenerant can be used to remove these unwanted metal ions. The resin can then be rinsed with an appropriate solvent or deionized water to remove any remaining traces of the conditioning agents, impurities, or unwanted metal ions. The pH of the resin can be adjusted using acidic or basic solutions to condition the resin and prepare it for selective metal ion binding. The resin is allowed to equilibrate with the solution it will be used in.

After complexing DOTA and Gadolinium, an intermediate product was obtained. The intermediate product included 2% to 4% metastable complexes by volume of non-metastable complexes.

An exposure step was performed. The intermediate product was exposed to each resin in a batchwise design for 15 minutes. Without wishing to be bound by a single theory, it is believed that 5, 6, 7, 8, 9, 10, 12, 15, 20, 25, 30 or up to 35 minutes of exposure time is preferable. If the exposure time is too short, metastable complexes are not removed. If the exposure time is too long, non-metastable complexes can be caused to degrade into undesirable metastable complexes.

The intermediate product and resin was the added to a stirred vessel.

After exposure to the resin for 15 minutes, the resin was promptly separated from resulting product by filtration with a sintered glass filter.

It is contemplated that the step of exposure to the resin can be repeated.

As a final step, the pH of the product was adjusted to 6.5-8.0 with meglumine.

Particularly strong results were obtained with thiourea or iminodiacetic acid chelator resins and strong acid cation resins. Preferably, the strong acid cation resins will be in the H+ or meglumine+ counter ion form. When using resins in the H+ form, significant meglumine will be lost to the resin. However, the meglumine can simply be added back without forming metastable complexes. When using other resins a small amount of meglumine can be lost. However, the meglumine can be added back to the final product so that pH is suitable for injection into the human body and the content of meglumine is within specification.

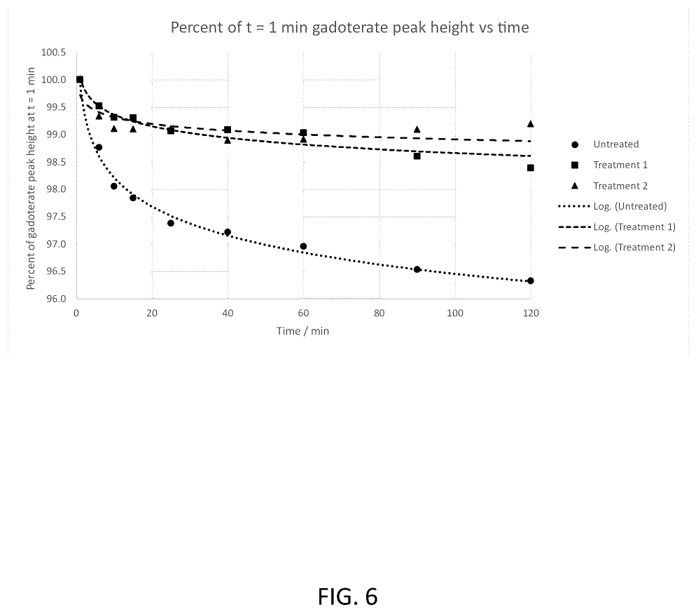

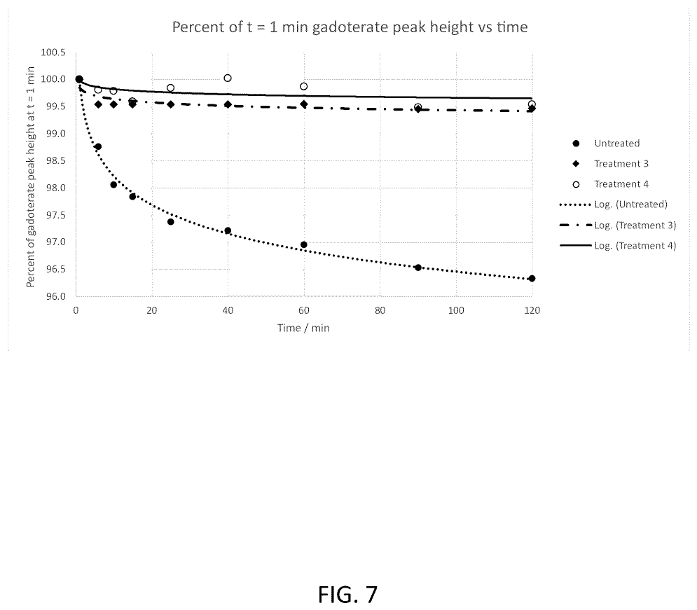

Results are summarized in FIGS. 6 and 7 and Table 3 below.

TABLE 3

| | Percentage of t = 1 min of UV peak heights (274 nm) | | | | |
|---|---|---|---|---|---|
| | AS13-117-1 | AS13-117-2 | AS13-117-6 | AS13-117-8 | |
| | Scale-up batch | Scale-up batch | Scale-up batch | Scale-up batch | AS13-24-5 |
| | treated w/ | treated w/ | treated w/ | treated w/ | Scale-up |
| | AG50W-X8-H+ | AG50W-X12- | AG50W-X8- | C150-MegH+ | batch |
| | (1 eq.) | H+ (1 eq.) | MegH+ (1 eq.) | (1 eq.) | untreated |
| Time post | Treatment 1 | Treatment 2 | Treatment 3 | Treatment 4 | Untreated |
| mix/min | AS13-116-1 | AS13-116-5 | AS13-116-6 | AS13-116-8 | AS13-96-1 |
| 1 | 100.0 | 100.0 | 100.0 | 100.0 | 100.0 |
| 6 | 99.5 | 99.3 | 99.5 | 99.8 | 98.8 |
| 10 | 99.3 | 99.1 | 99.5 | 99.8 | 98.1 |
| 15 | 99.3 | 99.1 | 99.5 | 99.6 | 97.8 |
| 25 | 99.1 | 99.1 | 99.5 | 99.8 | 97.4 |
| 40 | 99.1 | 98.9 | 99.5 | 100.0 | 97.2 |
| 60 | 99.0 | 98.9 | 99.5 | 99.9 | 97.0 |
| 90 | 98.6 | 99.1 | 99.5 | 99.5 | 96.5 |
| 120 | 98.4 | 99.2 | 99.5 | 99.5 | 96.3 |

The graphs shown in FIGS. 6 and 7 plot the Concentration of the gadoterate complex as measured by UV absorbance for the gadoterate molecule vs. time of exposure to Purolite® C150, Na+ strong acid cation resin over a period of 1 to 120 minutes. The Purolite® C150 resin is strong enough to dissociate gadolinium from metastable cages but too weak to dissociate gadolinium from fully coordinated cages. By comparing the gadoterate concentration by UV in each timepoint sample to the concentration of gadoterate in the T=1 minute sample, the amount of metastable cages in the product can be measured. The data shows that the untreated control sample has 250-700% more metastable complexes than the samples exposed to the resins listed Table 3 for 15 minutes in accordance with the methods disclosed herein.

It was unexpectedly found that exposure to the resins can be accomplished with very modest degradation of the lanthanide/chelate (gadoterate) molecules already in the most stable configuration. Only weaker cages are degraded under proper conditions.

Table 4 shows for HPLC results of gadoterate concentration before and after treatment with selected resins. All treatments are with 1.0 eq. of resin except as noted.

TABLE 4

| Sample name | Compound | Area counts | Area counts | Area counts | Area counts |
|---|---|---|---|---|---|
| Dotarem (T328A) | gadoterate | 10669.00 | 10658.47 | 10618.18 | 10893.75 |
| | meglumine | 2264.22 | 2250.32 | 2229.70 | 2256.09 |
| Clariscan (16364133) | gadoterate | 10753.90 | 10937.32 | | |
| | meglumine | 2294.17 | 2301.01 | | |
| Untreated control | gadoterate | 10593.47 | 10702.95 | | |
| (AS13-24-5) | meglumine | 2183.19 | 2214.88 | | |
| MTS9140, 0.1 eq. | gadoterate | 9326.70 | 9411.73 | | |
| (AS13-103-1) | meglumine | 2058.74 | 2084.82 | | |
| C104, Na+ | gadoterate | 10473.41 | 10485.79 | | |
| (AS13-103-2) | meglumine | 1847.29 | 1846.58 | | |
| AG50W-X8, H+ | gadoterate | 9924.97 | 9941.49 | | |
| (AS13-117-1) | meglumine | 2075.38 | 2069.89 | | |
| AG50W-X12, H+ | gadoterate | 10276.03 | 10313.26 | | |
| (AS13-117-2) | meglumine | 2150.22 | 2141.43 | | |
| MTS9140 | gadoterate | 8702.43 | 8810.02 | | |
| (AS13-117-3) | meglumine | 1978.31 | 1994.83 | | |
| AG50W-X8, Na+ | gadoterate | 10482.67 | 10546.59 | | |
| (AS13-117-4) | meglumine | 1434.55 | 1376.45 | | |
| C150, Na+, 0.1 eq. | gadoterate | 10641.32 | 10725.90 | | |
| (AS13-117-5) | meglumine | 2056.12 | 2033.06 | | |
| Dotarem (T328A) | gadoterate | 11086.89 | 11062.54 | 11161.58 | 11034.03 |
| | meglumine | 2386.63 | 2342.20 | 2388.84 | 2360.60 |
| Untreated control | gadoterate | 10932.93 | 10812.77 | | |
| (AS13-24-5) | meglumine | 2298.88 | 2281.37 | | |
| AG50W-X8, Meg.+ | gadoterate | 10699.31 | 10588.37 | | |
| (AS13-117-6) | meglumine | 2262.92 | 2236.62 | | |
| UCW3126, Meg.+ | gadoterate | 10714.61 | 10711.26 | | |
| (AS13-117-7) | meglumine | 2261.55 | 2276.49 | | |
| C150, meg. + | gadoterate | 10475.99 | 10375.23 | | |
| (AS13-117-8) | meglumine | 2217.51 | 2218.73 | | |
| MTS9140, 0.1 eq. | gadoterate | 10516.76 | 10681.91 | | |
| (AS13-117-9) | meglumine | 2259.34 | 2302.36 | | |
| | Area | Correction | Corr. area | % of | |
| Sample name | counts ave. | factor | counts ave. | comparitor | Comparitor |
| Dotarem (T328A) | 10709.85 | 0.9742 | 10433.61 | 100.0 | T328A |
| | 2250.08 | 0.9742 | 2192.05 | 100.0 | |
| Clariscan (16364133) | 10845.61 | 0.9780 | 10606.84 | 101.7 | T328A |
| | 2297.59 | 0.9780 | 2247.01 | 102.5 | |
| Untreated control | 10648.21 | 0.9897 | 10538.88 | 101.0 | T328A |
| (AS13-24-5) | 2199.04 | 0.9897 | 2176.46 | 99.3 | |
| MTS9140, 0.1 eq. | 9369.22 | 0.9873 | 9250.24 | 87.8 | AS13-24-5 |
| (AS13-103-1) | 2071.78 | 0.9873 | 2045.47 | 94.0 | |
| C104, Na+ | 10479.60 | 0.9906 | 10380.98 | 98.5 | AS13-24-5 |
| (AS13-103-2) | 1846.94 | 0.9906 | 1829.55 | 84.1 | |
| AG50W-X8, H+ | 9933.23 | 1.0248 | 10179.26 | 96.6 | AS13-24-5 |
| (AS13-117-1) | 2072.64 | 1.0248 | 2123.97 | 97.6 | |
| AG50W-X12, H+ | 10294.65 | 0.9885 | 10175.95 | 96.6 | AS13-24-5 |
| (AS13-117-2) | 2145.83 | 0.9885 | 2121.08 | 97.5 | |
| MTS9140 | 8756.23 | 0.9988 | 8746.02 | 83.0 | AS13-24-5 |
| (AS13-117-3) | 1986.57 | 0.9988 | 1984.25 | 91.2 | |
| AG50W-X8, Na+ | 10514.63 | 0.9938 | 10449.01 | 99.1 | AS13-24-5 |
| (AS13-117-4) | 1405.50 | 0.9938 | 1396.73 | 64.2 | |
| C150, Na+, 0.1 eq. | 10683.61 | 0.9769 | 10436.88 | 99.0 | AS13-24-5 |
| (AS13-117-5) | 2044.59 | 0.9769 | 1997.37 | 91.8 | |
| Dotarem (T328A) | 11086.26 | 0.9561 | 10599.23 | 100.0 | T328A |
| | 2369.57 | 0.9561 | 2265.47 | 100.0 | |
| Untreated control | 10872.85 | 0.9923 | 10789.55 | 101.8 | T328A |
| (AS13-24-5) | 2290.13 | 0.9923 | 2272.58 | 100.3 | |
| AG50W-X8, Meg.+ | 10643.84 | 1.0002 | 10645.83 | 98.7 | AS13-24-5 |
| (AS13-117-6) | 2249.77 | 1.0002 | 2250.19 | 99.0 | |
| UCW3126, Meg.+ | 10712.94 | 0.9848 | 10549.59 | 97.8 | AS13-24-5 |
| (AS13-117-7) | 2269.02 | 0.9848 | 2234.42 | 98.3 | |
| C150, meg.+ | 10425.61 | 0.9831 | 10249.88 | 95.0 | AS13-24-5 |
| (AS13-117-8) | 2218.12 | 0.9831 | 2180.73 | 96.0 | |
| MTS9140, 0.1 eq. | 10599.34 | 0.9757 | 10342.05 | 95.9 | AS13-24-5 |
| (AS13-117-9) | 2280.85 | 0.9757 | 2225.49 | 97.9 | |

It is contemplated that a fixed bed design will be as effective as a batchwise design. The present disclosure has found that resin contact times of 5 to 35 minutes are most effective.

It is contemplated that for large scale production, a fixed bed design allows the ion exchange to be terminated more expeditiously.

### Example 2: Removal of Gadolinium Ions from Gd-DOTA Preparation/Excess DOTA with Semipermeable Membrane

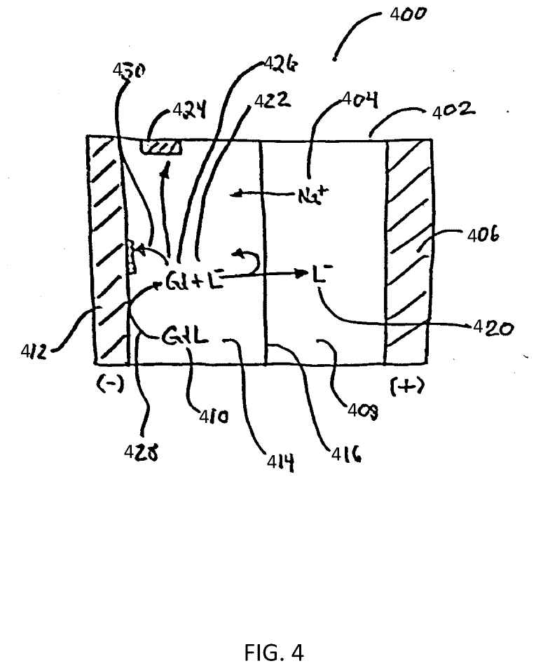

FIG. 4 illustrates a membrane purification system 400, comprising an electrolytic cell 402 containing an electrolyte 404 on the anode 406 side 408, for example NaCl, and a lanthanide-ligand complex 410 in aqueous solution, for example Gd-DOTA, on the cathode 412 side 414.

A semipermeable membrane 416 separates sides 414 and 408. The porosity of the semipermeable membrane 416 is chosen to allow free ligand 420 across membrane 416, but to exclude transport of the complex 410.

The semipermeable membrane purification process of example 4 can be performed without electrolysis. If the complex 410 is metastable, it has a nonzero thermodynamic stability constant, which means if one waits long enough, the metastable complex 410 will eventually decomplex 422 into free lanthanide ion and free ligand. The time for a metastable complex to disassociate is much shorter than the time for a fully coordinated complex to disassociate. If there is a lanthanide sequestration means, for example a solid state cationic resin 424, the free lanthanide can be removed from the solution before it complexes with any free ligand 420 present.

The semipermeable membrane purification process of example 4 can have the cationic resin 424 located on the anion side 408. Typically, the lanthanide ion 426 is small enough to pass through semipermeable membrane 416. The advantage in this arrangement is that the cationic resin 424 need not be in a solid state if the semipermeable membrane 416 excludes transport of the cationic resin 424 to cationic side 414. Although a cationic resin is used in this example, an anionic resin could also be used.

Time, heat, and pH can be used to challenge metastable complexes 410, encouraging them to disassociate. Preferably, the challenge does not cause stable complexes to disassociate. Electrolysis provides a quantifiable, and more rapid way to disassociate metastable complexes 410 and remove them differentially from a solution of fully coordinated complexes. When electrodes 406 and 412 are charged, metastable complex 410 is disassociated 428 on electrode 412. Lanthanide 426 is then deposited 430 on electrode 412.

All these semipermeable membrane purification procedures will remove heavy and transitional metal contaminants. The electrolysis method is preferred since metal contaminants can be metastably complexed with the ligand x20. Toxic metals removed by the present purification procedures include Sc, Y, La, Ce, Pr, Nd, Eu, Tb, Tm, Dy, Ho, and Eu. These metals have been found in commercially available, high purity gadolinium oxides. Terbium, Thulium, Europium and Lanthanum were found in the highest amounts, which were 0.42 mg/L, 0.17 mg/L, 0.17 mg/L, and 0.16 mg/L, respectively.

Generally, a membrane electrolysis cell comprises cathode and anode, which can be divided by a cation exchange membrane which prohibits negatively charged ion transport between the electrolytes. In this cell, the electrolyte solution surrounding the cathode is known as catholyte and the electrolyte near the anode is called anolyte. Catholyte electrolyte contains metal and chelating ligands, whereas anolyte electrolyte consists of sodium chloride.

During membrane electrolysis, Na+ ions from the anolyte solution diffuse and carry the current through the cation exchange membrane into the catholyte solution to complete the electrical circuit of the process.

### Example 5: Challenging Metastable Complexes with Magnetic Fields

The Curie temperature of gadolinium is 19° C. Below the Curie temperature gadolinium is ferromagnetic. In the ferromagnetic state, the gadolinium ions in the complex can be excited by an alternating magnetic field. Gadolinium excitation increases the kinetic energy of the ion in the complex, causing metastable complexes to disassociate. The disassociated metal ion can be removed by resin, semipermeable membrane sequestration, or electrolysis.

It should be noted that the terms “first”, “second”, and the like can be used herein to modify various elements. These modifiers do not imply a spatial, sequential, or hierarchical order to the modified elements unless specifically stated.

As used herein, the terms “a” and “an” mean “one or more” unless specifically indicated otherwise.

As used herein, the term “substantially” means the complete or nearly complete extent or degree of an action, characteristic, property, state, structure, item, or result. The exact allowable degree of deviation from absolute completeness can in some cases depend on the specific context. However, generally, the nearness of completion will be to have the same overall result as if absolute and total completion were obtained.

As used herein, the term “comprising” means including, but not limited to; the term “consisting essentially of” means that the method, structure, or composition includes steps or components specifically recited and can also include those that do not materially affect the basic novel features or characteristics of the method, structure, or composition; and the term “consisting of” means that the method, structure, or composition includes only those steps or components specifically recited.

As used herein, the term “about” is used to provide flexibility to a numerical range endpoint by providing that a given value can be “a little above” or “a little below” the endpoint. Further, where a numerical range is provided, the range is intended to include any and all numbers within the numerical range, including the end points of the range.

While the present disclosure has been described with reference to one or more exemplary embodiments, it will be understood by those skilled in the art, that various changes can be made, and equivalents can be substituted for elements thereof without departing from the scope of the present disclosure. In addition, many modifications can be made to adapt a particular situation or material to the teachings of the present disclosure without departing from the scope thereof. Therefore, it is intended that the present disclosure will not be limited to the particular embodiments disclosed herein, but that the disclosure will include all aspects falling within the scope of a fair reading of appended claims.

What is claimed is:

1. A method of preparing a stable lanthanide complex substantially free of metastable lanthanide complex and free lanthanide ion, the method comprising:
(i) complexing a macrocyclic chelator with a lanthanide metal in a suitable solvent to obtain a first solution of a metal complex;
(ii) challenging the metal complex to cause metastable complexes to form free lanthanide ion and free chelate to yield a second solution; and
(iii) removing the free lanthanide ion and free chelate from the second solution with a separation media.

2. The method of claim 1, wherein the separation media is an ionic acceptor resin that is cationic, and the method further comprises:
(iv) separating the ionic acceptor resin from the second solution to obtain a third solution that contains a metal complex containing less than 5 ppm free lanthanide ion and less than 0.5% metastable lanthanide complexes.

3. The method of claim 2, further comprising:
(v) removing excess chelate from the third solution by contacting one or more times with an anionic acceptor resin, whereby the excess chelate forms a complex with the anionic acceptor resin.

4. The method of claim 3, further comprising:
(vi) adding a chelator in a non-complexed form in the range from 0.005 to 0.05% w/v to the second or third solution to obtain a liquid pharmaceutical composition.

5. The method of claim 1, wherein the (ii) challenging further comprises adding an amount of meglumine to the first solution or cooling to room temperature at a pH below 2.5 and for a time of 2 hours to 24 hours.

6. The method of claim 1, wherein the (iii) removing is performed at a pH from 4.0 to 6.0.

7. The method of claim 1, wherein the (iii) removing is by contacting with an ionic acceptor resin to cause excess lanthanide to form a complex with the ionic acceptor resin.

8. The method of claim 7, wherein the ionic acceptor resin is selected from the group consisting of: iminodiacetic acid, ethylenediaminetetraacetic acid and diethylenetriaminepentaacetic acid, thiourea, strong acid cation, weak acid cation.

9. The method of claim 1, wherein the (iii) removing excess lanthanide is by a membrane separation that prevents passage of chelator and lanthanide complex through the membrane, a protein-based affinity chromatography process, or reverse osmosis.

10. The method of claim 1, wherein the lanthanide metal is at least one metal selected from the group consisting of: gadolinium, praseodymium, dysprosium, europium, and thulium and the macrocyclic chelator is at least one macrocyclic chelator selected from the group consisting of: DOTA, NOTA, DO3A, BT-DO3A, HP-DO3A and PCTA.

11. The method of claim 1, further comprising: alternating a magnetic field at a temperature below the Curie Temperature of the lanthanide metal during the (i) complexing.

12. A method for removing metastables from a macrocyclic chelator and lanthanide metal complex, the method comprising:
providing a solution containing the macrocyclic chelator and lanthanide metal complex;
challenging the macrocyclic chelator and lanthanide metal complex to cause metastable complexes to form free lanthanide ion and chelate to yield a solution;
removing excess lanthanide metals and macrocyclic chelator from the solution with a separation media that comprises a cationic or anionic acceptor resin configured with a highest affinity toward 3+ ions; and
separating the cationic or anionic acceptor resin from the solution to obtain a second solution that contains a metal complex substantially free of lanthanide ion and metastable lanthanide complexes.

13. The method of claim 12, further comprising: adjusting a pH of the solution to be suitable for injection in a human body with meglumine addition after the separating.

14. An injectable contrast agent solution for use in diagnostic imaging, the solution comprising:
a complex of a macrocyclic chelate and a lanthanide metal;
less than 0.5% metastable lanthanide complexes by volume of non-metastable lanthanide metal complexes in the lanthanide metal complex; and
less than 5 ppm free lanthanide ions by weight of lanthanide metal complexes.

15. The method of claim 12, wherein the challenging comprises:
applying the solution to an ion exchange resin configured to remove free lanthanide metals and dechelate metastable complexes from the solution for at least 5 minutes and at most 30 minutes.

16. The method of claim 15, wherein the ion exchange resin is a cation exchange resin.

17. The method of claim 15, wherein the ion exchange resin is a chelating resin selected from the group consisting of: thiourea, iminodiacetic acid, animinophosphonic acid, ethylenediaminetetraacetic acid and diethylenetriaminepentaacetic acid.

18. The method of claim 12, wherein the lanthanide metal chelate complex is gadoteric acid.

19. The method of claim 15, further comprising: controlling a temperature to be in a range of 10° C. to 50° C. while applying the solution to the ion exchange resin.

20. The method of claim 15, further comprising: applying the solution to the ion exchange resin in concentration of 0.1 to 1.0 equivalents.
